(19) United States
(12) Patent Application Publication
(10) Pub. No.: US 2014/0235848 A1
(43) Pub. Date: Aug. 21, 2014

(54) METHOD FOR MAKING A PRECURSOR OF L-FUCOSE FROM D-GLUCOSE

(71) Applicant: Glycom A/S, Kgs. Lyngby (DK)

(72) Inventors: Julien BOUTET, La Plaine sur Mer (FR); Nikolay KHANZHIN, Humlebaek (DK); Filippo BONACCORSI, Livorno (IT); Gyula DEKANY, Sinnamon Park (AU)

(73) Assignee: Glycom A/S, Kgs. Lyngby (DK)

(21) Appl. No.: 14/348,014

(22) PCT Filed: Sep. 28, 2012
(86) PCT No.: PCT/IB2012/055210

§ 371 Date: Mar. 27, 2014

(30) Foreign Application Priority Data
Sep. 30, 2011 (EP) 11183614.4

(51) Int. Cl. C07H 9/02; C07H 13/04; C07H 11/00; C07H 13/08

(52) U.S. Cl. CPC C07H 9/02; C07H 13/08; C07H 13/04; C07H 11/00
USPC 536/119; 536/115; 536/122; 536/124; 536/1.11

(57) ABSTRACT

A method that can be used to make a precursor of L-fucose from D-glucose that includes the steps of a) making a compound of formula (1) from D-glucose, formula (1) wherein R₁ is acyloxy, and Q is a group (a), (b), (c) or (d), formula (a), (b), (c) or (d) or wherein R₁ is OH, and Q is a group (e), (f) or (g); formula (e), (f) or (g) wherein R₂ is acyloxy and R₃ is a sulphonyl leaving group; and b) producing 6-deoxy-L-talose from the compound of formula (1) formed in step a), wherein the moiety is a highly lipophilic protecting group; compounds according to formula (1), and use of a compound according to formula (1) are provided.

### FIELD OF THE INVENTION

L-Monosaccharides or L-sugars, especially L-hexoses, are scarce in nature. Nevertheless, some L-hexoses are key building blocks in biologically important oligosaccharides, glycopeptides and other glycoside type derivatives among which L-fucose (6-deoxy-L-galactose) and L-rhamnose (6-deoxy-L-mannose) are best known.

Owing to their biological and medicinal properties and their scarcity in nature, chemists have developed synthetic processes or pathways for making L-sugars from abundant and cheap D-sugars. Generally, these synthetic pathways have included the extensive use of selective protective group manipulations and regio- and/or stereoselective functional group transformations such as S_{N}2-type inversions (epimerization), oxidation-reduction sequences, β-eliminations, additions to double bonds including C═O and/or C═C double bonds, and deoxygenations. These synthetic pathways have commonly included several steps, in which process intermediates have often needed to be isolated from reaction mixtures and purified prior to the next process steps.

For example, D-glucose has been converted into 6-deoxy-1,2-O-isopropylidene-β-L-talofuranose (compound F), a compound serving as precursor for modified nucleoside analogs (Zsoldos-Mády et al. Monatsh. Chem. 117, 1325 (1986), Hiebl et al. ibid. 121, 691 (1990)) and for chiral diphosphite ligands for asymmetric catalytic reactions (Diéguez et al. Chem. Eur. J. 7, 3086 (2001)). See the three pathways in Scheme 1 below. All three pathways have a common route from D-glucose to 3-O-acetyl-1,2-O-isopropylidene-α-D-allofuranose (compound A) in five steps. Compound A was then converted into the epoxide of formula E1 or E2 in four steps involving the introduction of a sulphonate leaving group in position 5 via regio- and chemoselective protective group manipulations, and the epoxides were then treated with LiAlH₄ to give compound F. All the three pathways have involved as many as ten elementary functional group transformations which have made each process cumbersome, inefficient and hence unattractive for industrial application.

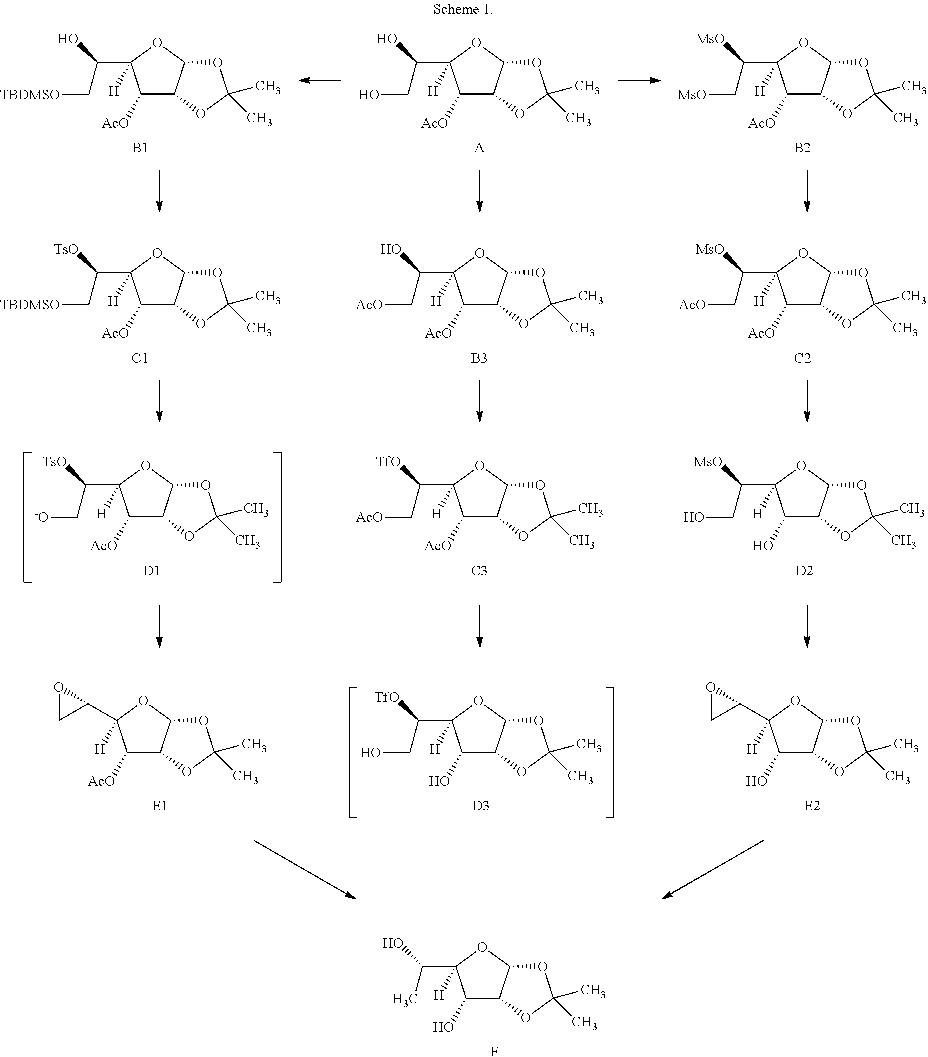

Although numerous synthetic processes have been developed to convert readily available cheap D-sugars into L-sugars, there has been a need for processes which take less time, require less reagents/solvents and/or provide better yields.

### SUMMARY OF THE INVENTION

The present invention provides a process for making L-fucose from D-glucose. In this process, better overall yields are obtained, and a simpler purification procedure can be used as compared with prior processes. As a result, the process can readily be carried out on a large scale, for efficient commercial production of L-fucose.

A first aspect of this invention relates to a method that can be used for making a precursor of L-fucose from D-glucose and that comprises steps of
a) making a compound of formula 1 from D-glucose,

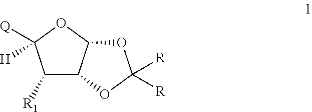

wherein the moiety

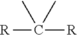

is a highly lipophilic protecting group, R₁ is acyloxy, and Q is a group (a), (b), (c) or (d)

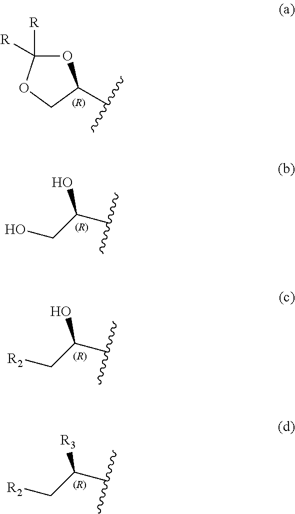

or when R₁ is OH, then Q is a group (e), (f) or (g)

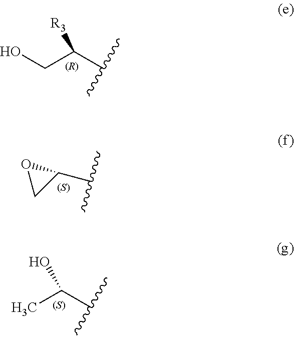

wherein R₂ is acyloxy, R₃ is a sulphonyl leaving group, and the moiety

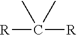

is as defined above,
and then b) converting the compound of formula 1 into 6-deoxy-L-talose.

A second aspect of this invention relates to the compounds of formula 1, above.

A third aspect of this invention relates to the use of a compound of formula 1 in the manufacture of 6-deoxy-L-talose or L-fucose from D-glucose.

### DETAILED DESCRIPTION OF THE INVENTION

In this invention, the term “highly lipophilic protecting group” preferably means a protecting group, such as a longer alkyl chain ketal group or a cyclic ketal group, for a compound that is a process intermediate. Such a protecting group makes the intermediate more lipophilic and thus more soluble in organic solvents. In preferred “highly lipophilic protecting groups”, the moiety

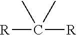

is a hydrocarbon group of at least 5 carbon atoms, especially wherein R individually is a C₂₋₆ alkyl or phenyl, or wherein the two geminal R groups together with the carbon atom to which they are attached form a C₅₋₈ cycloalkylidene, and particularly preferably wherein the two geminal R groups together with the carbon atom to which they are attached form a cyclopentylidene or a cyclohexylidene, most preferably a cyclohexylidene.

Herein, the term “alkyl”, unless otherwise stated, preferably means a linear or branched chain saturated hydrocarbon group with 1-6 carbon atoms, such as methyl, ethyl, n-propyl, i-propyl, n-butyl, i-butyl, s-butyl, t-butyl or n-hexyl.

Herein, the term “C₅₋₈ cycloalkylidene” preferably means a cycloalkylidene group optionally substituted with alkyl(s) wherein the cycloalkylidene group together with the optional substituent(s) has 5-8 carbon atoms, such as cyclopentylidene, cyclohexylidene, cycloheptylidene or 4,4-dimethyl-cyclohexylidene.

Herein, the term “acyloxy” means a univalent radical of an oxoacid represented by the formula R′—COO—, wherein R′ is H, alkyl or aryl (e.g., phenyl or naphthyl), such as acetoxy or benzoyloxy. Both alkyl and aryl groups can be substituted by one or more substituents selected from halogen, hydroxyl, alkyl, alkyloxy, nitro, amino, alkylamino, dialkylamino, thiol, alkylsulfanyl, aryl and aryloxy.

Herein, the term “sulphonate leaving group” means sulphonate esters which can be displaced by a nucleophile in a nucleophilic substitution reaction. More specifically, a sulphonate leaving group can be represented by the formula —OSO₂—Rₐ, wherein Rₐ means an alkyl group optionally substituted with one or more halogen atoms, preferably fluoro, a homoaromatic group selected from phenyl and naphthyl, or a 5-10 membered mono- or bicyclic heteroaromatic group having 1, 2 or 3 heteroatoms selected from O, N and S. The homo- and hetero-aromatic groups can be substituted with, for example, alkyl, halogen or nitro groups. Typical sulphonate leaving groups are mesylate (methanesulphonate), besylate (benzenesulphonate), tosylate (4-methylbenzenesulphonate), brosylate (4-bromobenzenesulphonate), nosylate (4-nitrobenzenesulphonate), triflate (trifluoromethanesulphonate), tresylate (2,2,2-trifluoroethanesulphonate) and 1-imidazolesulphonate.

The method of the first aspect of this invention, as shown below in Scheme 2, is simple and can be carried out in a generally straightforward manner as described below. Different compounds of formula 1 of the method are intermediates between D-glucose and 6-deoxy-L-talose as shown in Scheme 2.

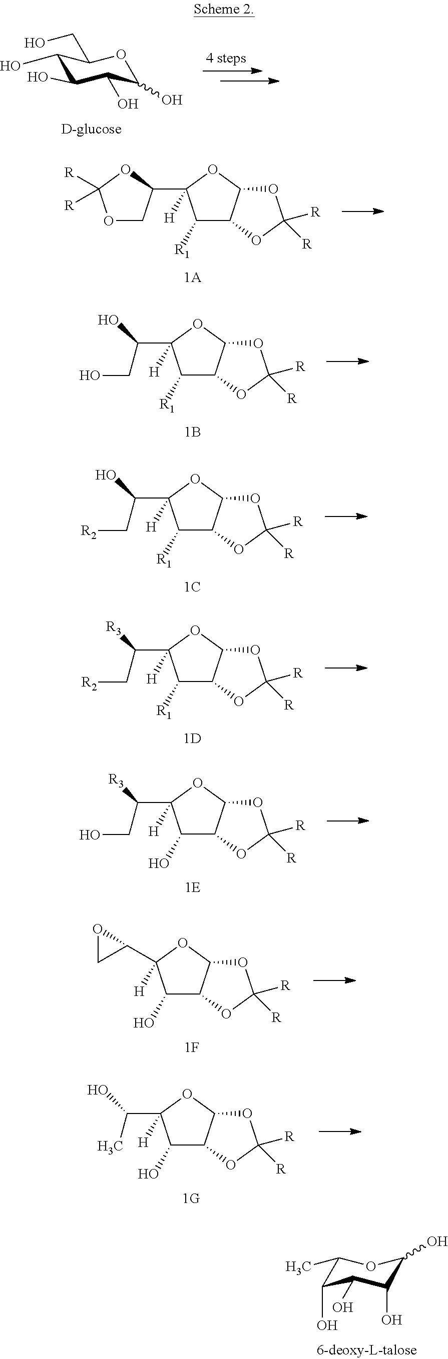

In carrying out the overall process of Scheme 2, any compound of formula 1 can be converted into a second compound of formula 1 without necessarily isolating the second compound before proceeding to convert the second compound into a third compound of formula 1. In this process, it is preferred that:
in each compound 1A-1G of formula 1 in Scheme 2, in the moiety

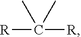

the two geminal R groups together with the carbon atom to which they are attached form a highly lipophilic protecting group that is a C₅₋₈ cycloalkylidene group, particularly preferably a cyclopentylidene or a cyclohexylidene group, and most preferably a cyclohexylidene group;
in each compound 1A-1D of formula 1 in Scheme 2, R₁ is acetoxy or benzoyloxy;
in compound 1C of formula 1 in Scheme 2, R₂ is acetoxy or benzoyloxy; and/or
in each compound 1D and 1E of formula 1 in Scheme 2, R₃ is mesyloxy or tosyloxy.

One embodiment of the compound of formula 1 is of formula 1G

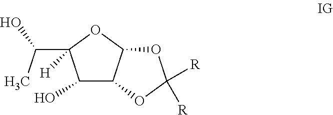

wherein

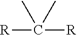

is as defined above,
   and is subjected to acidic hydrolysis in step b) in Scheme 2.

Another embodiment of the compound of formula 1 is of formula 1F

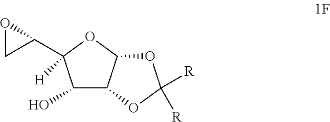

wherein

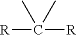

is as defined above,
   which can be treated with a reducing agent to product the compound of formula 1 G in Scheme 2.

Yet another embodiment of the compound of formula 1 is of formula 1E

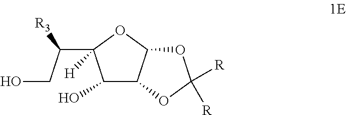

wherein

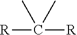

and R₃ are as defined above,
   which can be treated with a base to form the compound of formula 1F in Scheme 2.

A further embodiment of the compound of formula 1 is of formula 1 D

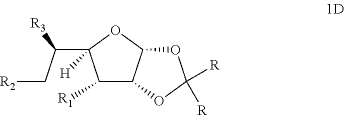

wherein

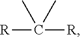

R₂ and R₃ are as defined above,
   which can be treated with a base to form the compound of formula 1E or 1F in Scheme 2.

A still further embodiment of the compound of formula 1 is of formula 1 D

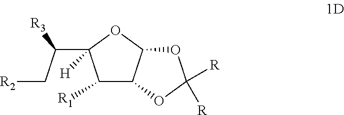

wherein

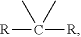

R₂ and R₃ are as defined above,
   which can be treated simultaneously or sequentially with a complex metal hydride reducing agent and a base to form the compound of formula 1 G in Scheme 2.

Yet another embodiment of the compound of formula 1 is of formula 1C

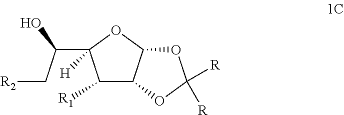

wherein

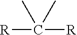

and R₂ are as defined above,
   which can be treated with a sulphonylation agent to form the compound of formula 1 D in Scheme 2.

Another embodiment of the compound of formula 1 is of formula 1B

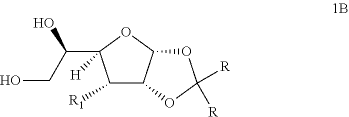

wherein

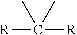

is as defined above,
   which can be subjected to selective 6-O-acylation to form the compound of formula 1C in Scheme 2.

Still another embodiment of the compound of formula 1 is of formula 1A

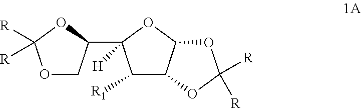

wherein

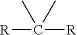

and R₁ are as defined above,
   which can be subjected to selective acidic hydrolysis to form the compound of formula 1B in Scheme 2.

Preferred conditions and reagents for carrying out the transformations above are given in the following description.

D-Glucose can be transformed into a compound of formula 1A in Scheme 2, above, in a multistep synthesis as shown in Scheme 3, below. In a first step, a 1,2:5,6-di-O-alkylidene-α-D-glucofuranose derivative 1AA, wherein

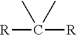

is as defined above, is formed by reacting the D-glucose with a keto derivative of formula R—C(═O)—R (such as cyclohexanone) or with the corresponding dialkyl acetal, preferably dimethyl acetal, under conventional acid catalysis conditions. The 3-OH group of the derivative 1AA is then oxidized in a conventional manner in a second step, to produce a corresponding ulose derivative 1AB, wherein

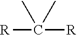

is as defined above. A suitable oxidizing agent can be, e.g., a chromium(VI) reagent (CrO₃-pyridine complex, Jones reagent, PCC, pyridinium dichromate, trimethylsilyl chromate, etc.), MnO₂, RuO₄, CAN, TEMPO, or DMSO in combination with DCC, Ac₂O, oxalyl chloride, tosyl chloride, bromine or chlorine. The ulose derivative 1AB is then reduced in a third step to a compound 1AC, wherein

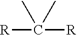

is as defined above. The reduction step can be carried out in a conventional manner with a reducing complex aluminium hydride or reducing complex borohydride, such as LiBH₄, KBH₄, Ca(BH₄)₂, Zn(BH₄)₂, tetrabutylammonium borohydride, LiAlH₄, NaAlH₄, KAlH₄ or Mg(AlH₄)₂. Compound 1AC can then be acylated in a conventional manner to form a compound of formula 1A in Scheme 2.

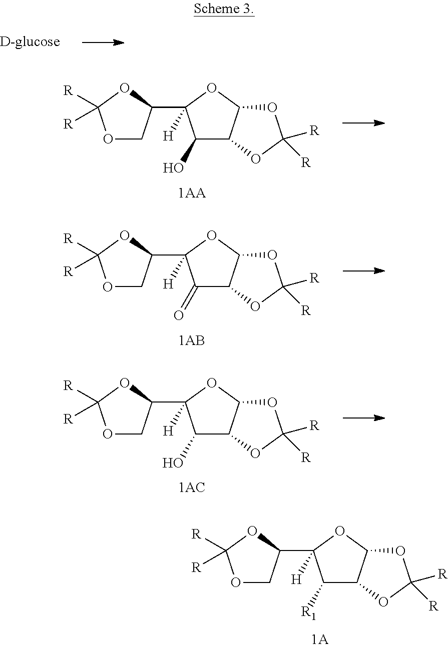

A compound of formula 1B in Scheme 2 can be made from a compound of formula 1A in Scheme 2 by selective acidic hydrolysis. In selective acidic hydrolysis, only the ketal protective group of the glycolic residue (i.e. 5,6-position) is removed while other acid labile protective groups, such as an acyloxy group or ketal in the 1,2-position remains intact. This is because acyloxy groups and ketals in the 1,2-position require stronger acidic conditions and/or longer reaction times to be deprotected than does a ketal in the 5,6-position; thus selective removal of the ketal in the 5,6-position can be easily accomplished because one can easily distinguish which deprotective condition(s) affect(s) the cyclic ketal in the terminal 5,6-position while leaving the acyloxy groups or cyclic ketal in the 1,2-position intact. Water, besides being the reagent, can serve as a solvent. Organic protic or aprotic solvents, which are miscible fully or partially with water such as C₁-C₆ alcohols, acetone, THF, dioxane, ethyl acetate or MeCN, can also be used in admixture with water. Suitable protic acids include acetic acid, trifluoroacetic acid, hydrochloric acid, formic acid, sulphuric acid, perchloric acid, oxalic acid, p-toluenesulfonic acid, benzenesulfonic acid and cation exchange resins, which can be present in from catalytic amounts to large excess. The acid hydrolysis can be carried out at temperatures between 20° C. and reflux, and completion of the reaction can take from about 1 hour to 3 days depending on the temperature, concentration and pH, used. Preferably, mild hydrolysis conditions with 60-80% acetic acid are used in this step. The resulting compound of formula 1B can be used either as the pure compound or as the crude reaction product in the next step.

A compound of formula 1C in Scheme 2 can be made from a compound of formula 1B in Scheme 2 by selective acylation in position 6. Selective acylation means that only the 6-OH group is acylated while the 5-OH group remains intact. Selective 6-O-acylation can be carried out with conventional acylating agents such as acyl halides, anhydrides or active esters in the presence of, for example, pyridine, triethylamine or diisopropyl ethylamine using organic solvents such as DCM, chloroform, THF, dioxane, acetonitrile or a mixture thereof at 20-80° C. to yield a compound of formula 1C. Preferably, acetic anhydride or benzoyl chloride is used in this step. The resulting compound of formula 1C can be used either as the pure compound or as the crude reaction product in the next step.

A compound of formula 1D in Scheme 2 can be made from a compound of formula 1C in Scheme 2 by sulphonylation. The reaction involves treatment of the alcohol of formula 1C with a slight excess of sulphonylating agent (≈1.5-3 equiv.) with or without added base, typically in an aprotic solvent such as toluene, THF, DCM, chloroform, dioxane, acetonitrile, chlorobenzene, ethylene dichloride, DMF, N-methylpyrrolidone, or mixtures thereof. As the sulphonylating agent, a conventional activated sulphonyl derivative can be used such as a halogenide or anhydride, wherein the sulphonyl group is of the formula —SO₂—Rₐ (see above). Typical sulphonylating agents include mesyl chloride, besyl chloride, tosyl chloride and trifluoromethanesulphonic anhydride. Preferably, mesyl or tosyl chloride are used. A tertiary amine base such as pyridine, substituted pyridine (such as dimethylamino-pyridine), N,N-dimethylaniline, triethyl amine, Hunig's base, and the like is preferably added to scavenge the liberated acid by-product, particularly pyridine, substituted pyridine, or N,N-dimethylaniline. The resulting compound of formula 1D can be used either as the pure compound or as the crude reaction product in the next step.

A compound of formula 1E in Scheme 2 can be made from a compound of formula 1D by treatment with a base. The term “base” means alkali metal or alkaline-earth metal hydroxides, alkoxides and carbonates, such as LiOH, NaOH, KOH, Mg(OH)₂, Ca(OH)₂, Ba(OH)₂, NaOMe, NaOEt, KO^{t}Bu, Li₂CO₃, Na₂CO₃, NaHCO₃, K₂CO₃ or BaCO₃. Strong basic ion exchange resins and tetraalkylammonium hydroxides can also be used. Any conventional solvent can be used that is not susceptible to nucleophilic attack by hydroxides or alkoxides. Typically, alkoxides can be added in C₁₋₄ alcohols at 20-100° C. to the reaction mixture. Carbonates and hydroxides can be added in water, alcohol or water-organic solvent mixtures, in homogeneous or heterogeneous reaction conditions at temperatures varying from 0-100° C. The resulting compound of formula 1E can be used either as the pure compound or as the crude reaction product in the next step.

A compound of formula 1E in Scheme 2 can then be treated with a base to form an epoxide of formula 1F. This reaction can be carried out in the same manner as described above for making a compound of formula 1E from a compound of formula 1D, and indeed, the epoxide of formula 1F can be made in the same pot as the compound of formula 1E without isolating the compound of formula 1E.

Reductive opening of the epoxide ring of the compound of formula 1F in Scheme 2 readily gives a compound of formula 1G. As the reducing agent in this step can be used a complex metal hydride having the formula MₓM′_{y}Hₙ, where M is an alkali or alkaline-earth metal cation or a cation complex and M′ is a metal or metalloid, preferably boron or aluminium. Typical borohydrides and aluminium hydrides include LiBH₄, KBH₄, Ca(BH₄)₂, Zn(BH₄)₂, tetrabutylammonium borohydride, NaBH(OMe)₃, NaBH₃NMe₂, NaBH₃NH^{t}Bu, tetrabutylammonium triacetoxyborohydride, LiBHEt₃, lithium or potassium tris(sec-butyl)borohydride, KBHPh₃, sodium cyanoborohydride, tetrabutylammonium cyanoborohydride, LiAlH₄, NaAlH₄, KAlH₄, Mg(AlH₄)₂, LiAlH(OMe)₃, LiAlH(OEt)₃, LiAlH₂(OEt)₂, LiAlH(O^{t}Bu)₃, LiAlH(OCEt₃)₃ or NaAlH₂(OCH₂CH₂OMe)₂. This reaction can be suitably carried out in an aprotic solvent that does not contain a functional group which is susceptible to hydride attack (such as an ester, keto or halogen group). Suitable solvents include ether type solvents such as diethyl ether, diisopropyl ether, THF or dioxane, and hydrocarbons, preferably aromatic hydrocarbons such as benzene, toluene, xylene, and mixtures thereof. When a borohydride reducing agent is used, water or C₁₋₄ alcohols such as methanol, ethanol, isopropanol, or mixtures thereof also can be used as the solvent preferably water or aqueous isopropanol.

Preferably, the compound of formula 1 D in Scheme 2 is treated simultaneously with the complex metal hydride reducing agent and the base, described above, to give the compound of formula 1G in Scheme 2. The reaction conditions described above for use with these reagents when they are used separately, can be used with these reagents when they are used together.

A compound of formula 1G in Scheme 2 can be readily converted into 6-deoxy-L-talose by acidic hydrolysis as described above. Water, besides being the reagent, can serve as a solvent. Protic acids, such as acetic acid, trifluoroacetic acid, HCl, formic acid, sulphuric acid, perchloric acid, oxalic acid, p-toluenesulfonic acid, benzenesulfonic acid, cation exchange resins, etc., can be used in amounts ranging from catalytic to a large excess. Temperatures between 20° C. and reflux can be used for periods of 1 hour to 3 days, depending on temperature, concentration and pH. Preferably, HCl and organic acids, particularly aqueous solutions of acetic acid, formic acid, chloroacetic acid or oxalic acid, or cation exchange resins, are used at a temperature in the range of 40-90° C., preferably 40-75° C. (Zsoldos-Mády et al. Monatsh. Chem. 117, 1325 (1986).

Optionally, 6-deoxy-L-talose can be epimerized in the presence of molybdic acid to yield L-fucose (Defaye et al. Carbohydr. Res. 126, 165 (1984); Hricovíniová Tetrahedron: Asymmetry 20, 1239 (2009), WO 2011/144213).

Surprisingly, the synthetic transformations of Scheme 2, described above, result in improved yields of the process intermediates of formulae 1A-1G as compared to prior transformations in which isopropylidene was used as the

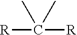

moiety, especially where extractive work-up procedures were needed to isolate such process intermediates. The ketal protective group having longer alkyl chains or the cyclic ketal protective group make the compounds of formula 1 more lipophilic and thus more soluble in organic solvents. This feature allows the use of smaller volumes of organic solvents and/or a smaller number of purification extractions, rendering the processes of Scheme 2 more efficient, quicker and more cost-effective, especially in large or industrial scale operations.

Additionally, the process intermediates of formulae 1A-1G are preferably crystalline materials. Crystallization or recrystallization is one of the simplest and cheapest methods to isolate a product from a reaction mixture, separate it from contaminants and obtain the pure substance. Isolation or purification that uses crystallization makes the whole technological process robust and cost-effective, and thus advantageous and attractive compared to other procedures.

A second aspect of the process of invention relates to the compounds of formula 1. The compounds of formula 1 can be crystalline solids, oils, syrups, precipitated amorphous material or spray dried products, but they are preferably crystalline. If crystalline, the compounds of formula 1 can exist either in anhydrous or hydrated crystalline forms, incorporating one or more molecules of water into their crystal structures. Similarly, compounds of formula 1 can exist as crystalline substances incorporating ligands such as organic molecules and/or ions into their crystal structures. The preferred compounds of formula 1 are those in which the two geminal R-groups together with the carbon atom to which they are attached form a C₅₋₈ cycloalkylidene group, particularly a cyclohexylidene group, and thereby are crystalline. Particularly preferred are the compounds of formula 1, in which the R₃ sulphonyl leaving group is mesylate, besylate, tosylate, triflate, nosylate, brosylate or tresylate, most preferably mesylate and tosylate, and in which the R₂ acyloxy group is acetoxy or benzoyloxy.

Other features of the invention will become apparent in view of the following exemplary embodiments which are illustrative but not limiting of the invention.

### EXAMPLES

### Example 1

1,2:5,6-di-O-cyclohexylidene-α-D-allofuranose (a compound of formula 1AC,

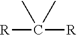

is cyclohexylidene)

To a solution of sodium bicarbonate (790 mg) in water (100 mL), acetone (100 mL), ruthenium dioxide hydrate (390 mg), sodium bromate (11.1 g) and 1,2:5,6-di-O-cyclohexylidene-α-D-glucofuranose (50 g) were added portionwise. The reaction mixture was stirred for 9 h at 23° C., then isopropanol (5 mL) was added and the mixture was stirred for further 3 h at 25° C. After filtration of the ruthenium dioxide, acetone was evaporated from the mixture and dichloromethane (100 mL) was added. The reaction mixture was then cooled to 10° C., then 25% NaOH solution (2 mL) was added followed by a solution of sodium borohydride (2.2 g) in 0.5% NaOH (10 mL). The emulsion was stirred for 30 min at 10-15° C., then the DCM phase was separated and the aqueous phase was washed with 200 mL DCM. The combined DCM phases were evaporated to afford 44.3 g white solid (88%). The compound can be recrystallized from n-hexane.

¹H NMR (CDCl₃, 300 MHz): δ=5.82 (d, 1H, H-1), 4.60 (m, 1H, H-2), 4.29 (m, 1H, H-5), 4.06-3.98 (m, 3H, H-3, H-4, H-6a), 3.79 (m, 1H, H-6b), 2.58 (m, 1H, OH-3), 1.78-1.22 (m, 10H, CH₂ cyclohexylidene). M.p.: 124-126° C.

### Example 2

3-O-acetyl-1,2:5,6-di-O-cyclohexylidene-α-D-allofuranose (a compound of formula 1A,

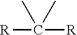

is cyclohexylidene, R₁ is acetoxy)

Acetic anhydride (1.3 eq.) was slowly added at 0° C. to a solution of 1,2:5,6-di-O-cyclohexylidene-α-D-allofuranose (146.9 mmol) in dichloromethane (200 mL) and pyridine (1.28 eq.). The reaction mixture was heated under reflux of DCM for 1 h, then cooled to 10° C. and 37% HCl solution (0.37 eq.) was slowly added. The DCM phase was separated and the aqueous phase was washed with 100 mL of DCM. The combined DCM phases were evaporated to afford an oily syrup. Yield: 95%.

¹H NMR (CDCl₃, 300 MHz): δ=5.79 (d, 1H, H-1), 4.85-4.76 (m, 2H, H-2, H-3), 4.21 (m, 1H, H-5), 4.11-3.99 (m, 2H, H-4, H-6a), 3.82 (m, 1H, H-6b), 2.02 (s, 3H, Ac), 1.78-1.24 (m, 10H, CH₂ cyclohexylidene).

### Example 3

3-O-acetyl-1,2-O-cyclohexylidene-α-D-allofuranose (a compound of formula 1B,

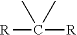

is cyclohexylidene, R₁ is acetoxy)

Water (20 mL) was added to a solution of 3-O-acetyl-1,2-5,6-di-O-cyclohexylidene-α-D-allofuranose (117.0 mmol) in acetic acid (80 mL), and the reaction mixture was heated at 80° C. for 2 h. The reaction was evaporated to dryness and coevaporated with toluene (2×50 mL). The oily syrup was purified by column chromatography (hexane/EtOAc: 1/1) to afford an oily syrup. Yield: 80%

¹H NMR (CDCl₃, 300 MHz): δ=5.81 (d, 1H, H-1), 4.85-4.78 (m, 2H, H-2, H-3), 4.17 (m, 1H, H-4), 3.79 (m, 1H, H-5), 3.58 (m, 1H, H-6a), 3.47 (m, 1H, H-6b), 2.03 (s, 3H, Ac), 1.78-1.24 (m, 10H, CH₂ cyclohexylidene).

### Example 4

### Compounds of Formula 1D

Benzoyl chloride or acetic anhydride (1 eq.) was slowly added to 3-O-acetyl-1,2-O-cyclohexylidene-α-D-allofuranose (10.1 mmol) in dichloromethane (15 mL) and pyridine (5 eq.) at −5° C., and the mixture was stirred at 0° C. for 3 h. Mesyl or tosyl chloride (1.3 eq.) was then added to the reaction mixture. After having been stirred for 12 h at 45° C., the reaction mixture was cooled to 0° C. and water (10 mL) was added followed by slow addition of 37% HCl solution (1 eq.). The DCM phase was separated and the aqueous phase was washed with 30 mL of DCM. The combined DCM phases were washed with saturated sodium bicarbonate (20 mL) and brine (20 mL) and then evaporated to afford an oily syrup. The compound can be crystallized from a mixture of ethyl acetate and cyclohexane.

### a) 3-O-acetyl-6-O-benzoyl-5-O-mesyl-1,2-O-cyclohexylidene-α-D-allofuranose

Yield: 49%. ¹H NMR (CDCl₃, 300 MHz): δ=8.02 (m, 2H, Bz), 7.59 (m, 1H, Bz), 7.42 (m, 2H, Bz), 5.82 (d, 1H, H-1), 5.21 (m, 1H, H-5), 4.95-4.84 (m, 2H, H-2, H-3), 4.62 (m, 1H, H-6a), 4.42-4.38 (m, 2H, H-4, H-6b), 3.02 (s, 1H, Ms), 2.03 (s, 3H, Ac), 1.77-1.21 (m, 10H, CH₂ cyclohexylidene). M.p.: 125-133° C.

### b) 3,6-di-O-acetyl-5-O-mesyl-1,2-O-cyclohexylidene-α-D-allofuranose

Yield: 57%. ¹H NMR (CDCl₃, 300 MHz): δ=5.82 (d, 1H, H-1), 5.06 (m, 1H, H-5), 4.86-4.81 (m, 2H, H-2, H-3), 4.43 (m, 1H, H-6a), 4.29 (m, 1H, H-4), 4.09 (m, 1H, H-6b), 3.02 (s, 1H, Ms), 2.08 (s, 3H, Ac), 2.03 (s, 3H, Ac), 1.77-1.25 (m, 10H, CH₂ cyclohexylidene). M.p.: 126-134° C.

### c) 3-O-acetyl-6-O-benzoyl-5-O-tosyl-1,2-O-cyclohexylidene-α-D-allofuranose

Yield: 48%. ¹H NMR (CDCl₃, 300 MHz): δ=7.94 (d, 2H, Tos), 7.75 (d, 2H, Tos), 7.56 (t, 1H, Bz), 7.42 (t, 2H, Bz), 7.21 (d, 2H, Bz), 5.55 (d, 1H, H-1), 5.13 (m, 1H, H-5), 4.92-4.84 (m, 2H, H-2, H-3), 4.51 (m, 1H, H-6a), 4.37-4.34 (m, 2H, H-4, H-6b), 2.33 (s, 1H, Tos), 2.07 (s, 3H, Ac), 1.70-1.30 (m, 10H, CH₂ cyclohexylidene). M.p.: 129-134° C.

### Example 5

6-deoxy-1,2-O-cyclohexylidene-β-L-talofuranose (a compound of formula 1 G,

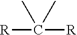

is cyclohexylidene)

Sodium hydroxide (4 eq.) in MeOH (1.5 mL) was slowly added at 0° C. to a solution of 3,6-di-O-acetyl-5-O-mesyl-1,2-O-cyclohexylidene-α-D-allofuranose (1 g) in 1,2-dimethoxyethane (4 mL). After 5 min. TLC showed formation of a new compound which proved to be 5-O-mesyl-1,2-O-cyclohexylidene-α-D-allofuranose [¹H NMR (CDCl₃, 300 MHz): δ=5.80 (d, 1H, H-1), 4.90 (m, 1H, H-5), 4.61 (m, 1H, H-2), 4.18 (m, 1H, H-3), 4.06-3.84 (m, 3H, H-4, H-6a, H-6b), 3.50 (s, 1H, OH-3), 3.18 (s, 1H, OH-6), 3.08 (s, 3H, CH₃ mesyl), 1.82-1.35 (m, 10H, CH₂ cyclohexylidene)]. After 30 min., saturated sodium bicarbonate solution (2 mL) was added followed by addition of 37% HCl solution (0.2 mL). Then dichloromethane (10 mL) was added and the phases were separated. The organic phase was evaporated to dryness to give 5,6-anhydro-1,2-O-cyclohexylidene-β-L-talofuranose [¹H NMR (CDCl₃, 300 MHz): δ=5.88 (d, 1H, H-1), 4.50 (m, 1H, H-2), 3.91 (m, 1H, H-3), 3.61 (m, 1H, H-4), 3.13 (m, 1H, H-5), 2.79 (m, 2H, H-6), 2.42 (s, 1H, OH-3), 1.78-1.31 (m, 10H, CH₂ cyclohexylidene)]. Sodium borohydride (0.7 eq.) was added to a solution of the crude product in 1,2-dimethoxyethane (3 mL). The reaction mixture was stirred for 12 h at 70° C., solvents were evaporated and the residue was partitioned between DCM and water. After separation the DCM was evaporated and the product was crystallized.

Yield: 75%. ¹H NMR (CDCl₃, 300 MHz): δ=5.80 (d, 1H, H-1), 4.54 (m, 1H, H-2), 3.89-3.81 (m, 2H, H-3, H-5), 3.58 (m, 1H, H-4), 1.78-1.32 (m, 10H, CH₂ cyclohexylidene), 1.22 (d, 3H, H-6). M.p.: 68-70° C.

### Example 6

### Partition Studies

1,2-O-Cyclohexylidene-6-deoxy-β-L-talofuranose (197 mg) was partitioned between water (10 mL) and methylene chloride (10 mL), layers separated and the residual amount of solutions in the separatory funnel were partitioned with extra amount of water (5 mL) and methylene chloride (5 mL). The combined organic phases were evaporated and dried in vacuo (50° C., <1 mbar, 1 hour) to give 140 mg of the title compound. The aqueous solution gave 53 mg after lyophilisation and drying in vacuo (50° C., <1 mbar, 2 hrs). Using the same procedure, partition between ethyl acetate and water provided 127 mg of the title compound from ethyl acetate phase and 70 mg from the aqueous phase.

Analogously, 1,2-O-isopropylidene-6-deoxy-β-L-talofuranose (150 mg) was partitioned between methylene chloride (24 mg) and aqueous phase (126 mg). Partition between ethyl acetate and water furnished 7 mg and 143 mg of the title compound, respectively. The results are summarized in the following table:

| | | Partition P | | Number of volumes of |
|---|---|---|---|---|
| Compound | Solvent system | (=[C]_{org}/[C]_{aq}) | logP | organic solvent* (yield) |
| 1,2-O-cyclohexylidene-6-deoxy- | CH₂Cl₂-water | 2.64 | 0.42 | 3 (98%) |
| β-L-talofuranose | | | | |
| 1,2-O-cyclohexylidene-6-deoxy- | ethyl acetate- | 1.81 | 0.26 | 3 (95.5%) |
| β-L-talofuranose | water | | | |
| 1,2-O-isopropylidene-6-deoxy-β- | CH₂Cl₂-water | 0.19 | 0.72 | 18 (95.6%) |
| L-talofuranose | | | | |
| 1,2-O-isopropylidene-6-deoxy-β- | ethyl acetate- | 0.16 | 0.79 | 21 (95.6%) |
| L-talofuranose | water | | | |

*To reach at least 95% of recovery using one volume of organic solvent each time relative to one volume of aqueous solution.

These results show that the highly lipophilic cyclohexylidene group on compound 1G, which is a protected 6-deoxy-L-talose derivative, resulted in compound 1G having a higher affinity to organic solvents as compared to aqueous media. This implies a surprisingly much higher solubility of compound 1G in organic solvents as compared to aqueous solvents, which tremendously facilitates its extraction into an organic solvent. By comparison, the corresponding isopropylidene compound had higher affinity to aqueous medium, therefore was highly soluble in aqueous solutions and almost insoluble in organic solvents.

1. A method that can be used to make a precursor of L-fucose from D-glucose, that comprises the steps of:
a) making a compound of formula 1 from D-glucose, 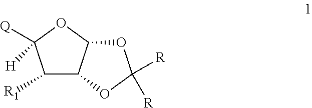
wherein R₁ is acyloxy, and Q is a group (a), (b), (c) or (d), 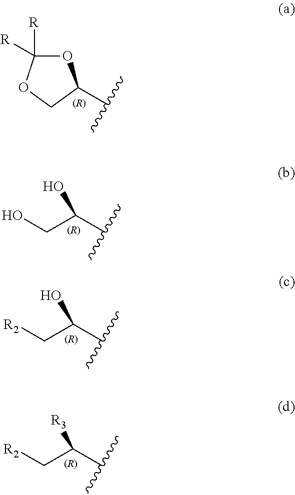
or wherein R₁ is OH, and Q is a group (e), (f) or (g) 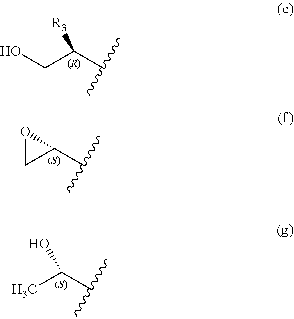
wherein R₂ is acyloxy and R₃ is a sulphonyl leaving group; and
b) producing 6-deoxy-L-talose from the compound of formula 1 formed in step a),
wherein the moiety 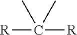
is a highly lipophilic protecting group.

2. A method according to claim 1, wherein step a) comprises making a compound of formula 1G 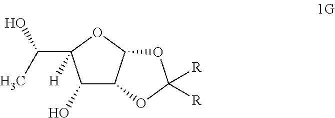
wherein 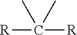
is as defined in claim 1, and
wherein step b) comprises acidic hydrolysis of the compound of formula 1G.

3. A method according to claim 2, wherein step a) comprises making a compound of formula 1F 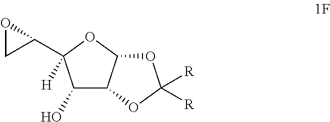
wherein 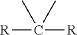
is as defined in claim 1,
and the treatment of the compound of formula 1F with a reducing agent to produce a compound of formula 1G.

4. A method according to claim 3, wherein step a) comprises making a compound of formula 1E 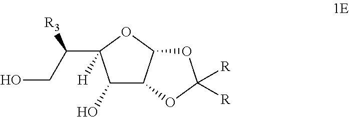
wherein 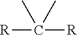
and R₃ are as defined in claim 1,
and the treatment of the compound of formula 1E with a base to form a compound of formula 1F.

5. A method according to claim 4, wherein step a) comprises making a compound of formula 1D 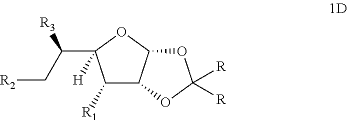
wherein 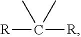
R₂ and R₃ are as defined in claim 1,
and the treatment of the compound of formula 1D with a base to form a compound of formula 1E or 1F.

6. A method according to claim 2, wherein step a) comprises making a compound of formula 1D 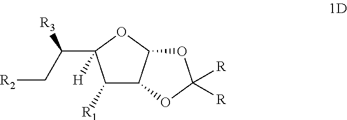
wherein 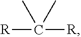
R₂ and R₃ are as defined in claim 1,
and treatment of the compound of formula 1D simultaneously or sequentially with a reducing agent and a base to form a compound of formula 1G.

7. A method according to claim 6, wherein step a) comprises making a compound of formula 1C 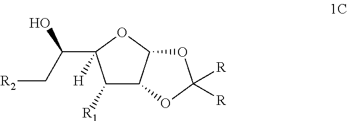
wherein 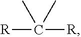
R₁ and R₂ are as defined in claim 1,
and treatment of the compound of formula 1C with a sulphonylating agent to form a compound of formula 1D.

8. A method according to claim 7, wherein step a) comprises making a compound of formula 1B 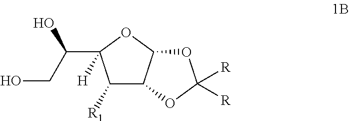
wherein 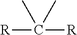
and R₁ are as defined in claim 1,
and subjecting the compound of formula 1B to selective 6-O-acylation to form a compound of formula 1C.

9. A method according to claim 8, wherein step a) comprises making a compound of formula 1A 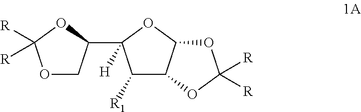
wherein 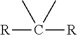
is as defined in claim 1,
and subjecting the compound of formula 1A to selective acidic hydrolysis to form a compound of formula 1B.

10. A method according to claim 9, wherein the step a) comprises making a compound of formula 1A from D-glucose by a method comprising the steps of:
a) reacting D-glucose with a keto derivative of formula R—C(═O)—R, or with the corresponding diallyl acetal thereof, under acid catalysis conditions to form the derivative of formula 1AA 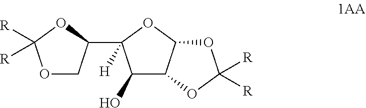
wherein 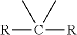
is as defined in claim 1;
b) oxidizing the derivative of formula 1AA to form the ulose derivative of formula 1AB 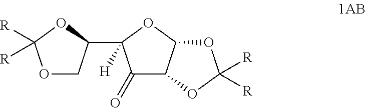
wherein 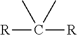
is as defined in claim 1;
c) reducing the ulose derivative of formula 1AB to form the derivative of formula 1AC 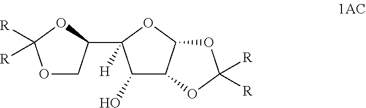
wherein 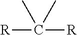
is as defined in claim 1; and then
d) acylating the derivative of formula 1AC to form the compound of formula 1A.

11. A method according to claim 1, wherein the moiety 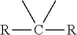
is a hydrocarbon group of at least 5 carbon atoms.

12. A method according to claim 11, wherein, in the moiety 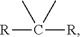
R is a C₂₋₆ alkyl or phenyl, or wherein the two geminal R groups together with the carbon atom to which they are attached form a C₅₋₈ cycloalkylidene.

13. A method according to claim 1, wherein R₃ is mesylate, tosylate, triflate, nosylate, brosylate or tresylate, and R₂ is acetoxy or benzoyloxy.

14. A compound according to formula 1 of claim 1.

15. A compound according to claim 14, wherein the moiety 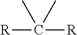
is a hydrocarbon group of at least 5 carbon atoms.

16. A compound according to claim 15, wherein, in the moiety 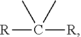
R is a C₂₋₆ alkyl or phenyl, or wherein the two geminal R groups together with the carbon atom to which they are attached form a C₅₋₈ cycloalkylidene.

17. A compound according to claim 14, wherein R₃ is mesylate, besylate, tosylate, triflate, nosylate, brosylate or tresylate, and R₂ is acetoxy or benzoyloxy.

18. A compound according to claim 14 that is isolated in crystalline form.

19. (canceled)

20. A method according to claim 3, wherein step a) comprises making a compound of formula 1D 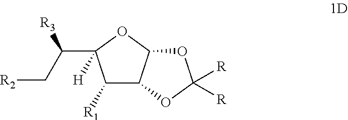
wherein 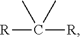
R₂ and R₃ are as defined in claim 1,
and the treatment of the compound of formula 1D with a base to form a compound of formula 1E or 1F.

21. A method according to claim 12, wherein the two geminal R groups together with the carbon atom to which they are attached form a cyclohexylidene.

22. A method according to claim 13, wherein R₃ is mesylate or tosylate.

23. A compound according to formula 1A of claim 1.

24. A compound according to formula 1B of claim 1.

25. A compound according to formula 1C of claim 1.

26. A compound according to formula 1D of claim 1.

27. A compound according to formula 1E of claim 1.

28. A compound according to formula 1F of claim 1.

29. A compound according to formula 1G of claim 1.

30. A compound according to claim 16, wherein the two geminal R groups together with the carbon atom to which they are attached form a cyclohexylidene.

31. A compound according to claim 17, wherein R₃ is mesylate or tosylate.
